(19) United States
(12) Patent Application Publication
(10) Pub. No.: US 2006/0155257 A1
(43) Pub. Date: Jul. 13, 2006

(54) Pharmaceutical delivery systems and methods for using same

(76) Inventor: David L Reynolds, Bromont (CA)

Correspondence Address: BERESKIN AND PARR, 40 KING STREET WEST, BOX 401, TORONTO, ON, M5H 3Y2, CA

(21) Appl. No.: 10/534,028

(22) PCT Filed: Nov. 7, 2003
(86) PCT No.: PCT/CA03/01713

§ 371 Date: Feb. 16, 2006

Related U.S. Application Data
(60) Provisional application No. 60/424,713, filed on Nov. 8, 2002.

(51) Int. Cl. A61M 5/32

(52) U.S. Cl. 604/414

(57) ABSTRACT

The present invention provides an assembly (10) for transferring a liquid between a vial (56) and a syringe (66). The assembly includes a housing (12) having a central portion (15), the housing being open at one end (19) and having a vial socket (16) at the other opposite end (17) adapted to receive and retain a vial having a penetrable closure (62). The housing further includes a sleeve (26) located within the central portion of the housing. The sleeve has a first portion (28a), a second portion (30) adjacent the first portion, and a shoulder (32) between the first portion and the second portion. The assembly also includes a protractible luer adaptor (14) with a central hub (44) having a first axial end and a second opposite axial end. The first axial end has a hollow piercing member (48) with a tip having an opening mounted thereon and the second axial end has an engaging member (38) for releasably engaging a syringe, the hollow piercing member, the central hub, and the engaging member being in fluid communication with one another. The protractible luer adaptor is longitudinally slidable within the sleeve between a retracted position where the hollow piercing member is substantially contained within the central portion of the housing and an advanced position where the tip of the hollow piercing member extends into the vial socket.

### FIELD OF THE INVENTION

The present invention generally relates to pharmaceutical delivery systems, and to methods for using same. More specifically, it relates to an assembly for transferring one or more components of a pharmaceutical composition from a pharmaceutical vial to a syringe or vice versa.

### BACKGROUND OF THE INVENTION

Traditionally, a syringe is filled manually by aspirating a liquid pharmaceutical component from a pharmaceutical vial having a neck with a penetrable closure into the syringe through a needle that penetrates the penetrable closure. The method of manually filling the syringe typically includes the following steps: (a) drawing air into the body of the syringe by pulling the syringe's plunger away from the needle end of the syringe until the volume of air in the body approximately equals the volume of pharmaceutical component to be loaded into the syringe; (b) carefully aligning the needle with the vial's penetrable closure and inserting the needle through the penetrable closure into the vial; (c) inverting the vial and forcing the air from the body of the syringe into the vial by advancing the syringe's plunger; (d) withdrawing the plunger to draw out the desired volume of the pharmaceutical component into the syringe; and (e) removing the needle from the vial.

This method suffers from various disadvantages. Firstly, the user is exposed to the unprotected needle tip, which can result in accidental stabbings or prickings. Secondly, if the user wishes to draw a large volume of the pharmaceutical component into the syringe (e.g., 10 cc) an equivalent volume of air must be forced into the vial. This can increase the pressure in the pharmaceutical vial to the point the pharmaceutical component may spray through the puncture point made in the penetrable seal and onto the user. These accidents can be particularly dangerous if the pharmaceutical component is unsafe to the user, for example with toxic oncology pharmaceuticals. Thirdly, the sterility of the needle may be compromised during the process of transferring the pharmaceutical component from the vial to the syringe.

Additionally, many pharmaceutical preparations must be distributed as two or more separate components (commonly a solid component and a liquid component in which the solid component should be reconstituted shortly before administration of the preparation although it could be two liquid components). Traditionally, this reconstitution includes the following steps: (a) providing a first component packaged in a pharmaceutical vial having a neck closed by a penetrable closure; (b) providing a second liquid component in a syringe; (c) injecting the second liquid component into the vial through the penetrable closure; (d) swilling the vial impaled on the syringe to dissolve, dilute or suspend the first component in the second component; and (e) aspirating the combined components back into the syringe. Alternatively, the two or more components may be liquid and require mixing just prior to administration. The mixing may be accomplished in an analogous manner. These methods suffer from many of the disadvantages described above.

There is a need for a pharmaceutical delivery system that can be used with standard pharmaceutical vials and syringes, is safe and easy to manipulate, and is economical to manufacture.

### SUMMARY OF THE INVENTION

In one aspect of the invention, the present invention provides for a device for transferring a pharmaceutical component from a vial to a syringe comprising:

a) a cylindrical housing having a central portion, a vial socket for receiving a pharmaceutical vial, a syringe socket for receiving a syringe;

b) a first cylindrical sleeve located within the central portion of the housing, the first sleeve having a smaller diameter than the housing, the first sleeve having an annular detent on its inner wall;

c) a second cylindrical sleeve located within the central portion of the housing having a diameter smaller than the first cylindrical sleeve, the second cylindrical sleeve located adjacent to the first cylindrical sleeve and between the first cylindrical sleeve and the vial socket, thereby forming an annular shoulder at the juncture between the two, the second cylindrical sleeve having a first plurality of spaces longitudinal ribs on its inner wall;

d) a protractible luer adaptor having a central hub with a second plurality of spaced longitudinal ribs on its outer surface and a flange at one end, a female luer lock having a thread at one end, and a cannula at the other end, the cannula, hub and female luer lock being in fluid communication, the second plurality of longitudinal ribs being sized and spaced to slidingly fit between the first plurality of longitudinal ribs on the inner wall of the second cylindrical sleeve;

whereby the protractible luer adaptor is longitudinally slidable within the first and second cylindrical sleeves between a retracted position where the flange engages the annular detent and the cannula is contained with the central portion of the housing to an advanced position where the flange abuts the annular shoulder and the cannula extends into the vial socket.

### BRIEF DESCRIPTION OF THE DRAWINGS

For a better understanding of the present invention and to show more clearly how it may be carried into effect, reference will now be made, by way of example, to the accompanying drawings in which:

FIG. 1 is a side elevational view of a housing according to one aspect of the present invention;

FIG. 2 is a side elevational view of a housing according to a further aspect of the invention;

FIG. 3 is a side elevational view of a housing according to a further aspect of the invention;

FIG. 4 is a side elevational view of a pharmaceutical transfer assembly in a retracted or “unactivated” position according to one aspect of the present invention;

FIG. 5 is a side elevational view of the pharmaceutical transfer assembly shown in FIG. 4 in an advanced or “activated” position;

FIG. 6 is an exploded side elevational view of a pharmaceutical delivery system according to one aspect of the present invention;

FIGS. 7-11 illustrate successive stages in deployment of a pharmaceutical delivery system according to a further aspect of the present invention to transfer a fluid pharmaceutical component from a prepackaged pharmaceutical vial to a syringe;

FIG. 12 is a side elevational view of a pharmaceutical transfer assembly in a retracted or “inactivated” position according to a further aspect of the present invention;

FIG. 13 is a side elevational view of the pharmaceutical transfer assembly shown in FIG. 12 in an advanced or “activated” position;

FIG. 14 is an exploded side elevational view of a pharmaceutical delivery system according to a further aspect of the present invention;

FIGS. 15-20 illustrate successive stages in the deployment of a pharmaceutical delivery system according to a further aspect of the present invention to reconstitute a multi-component pharmaceutical;

FIGS. 21-25 illustrate successive stages in the deployment of a pharmaceutical delivery system in accordance with another embodiment of the present invention which utilizes a two piston syringe system;

FIGS. 26-31 illustrate successive stages in the deployment of a pharmaceutical delivery system using the syringe system of FIG. 21 to reconstitute a multi-component pharmaceutical;

FIG. 32 is a side elevational view of a pharmaceutical transfer assembly in a retracted or “unactivated” position according to a still further aspect of the present invention; and

FIG. 33 is a side elevational view of a pharmaceutical transfer assembly as shown in FIG. 32 in an advanced or “activated” position.

### DETAILED DESCRIPTION OF THE INVENTION

The pharmaceutical transfer assembly described below is adapted to be used with a standard pharmaceutical vial and syringe. Such standard vials and syringes are well known in the art, but examples will be described here briefly.

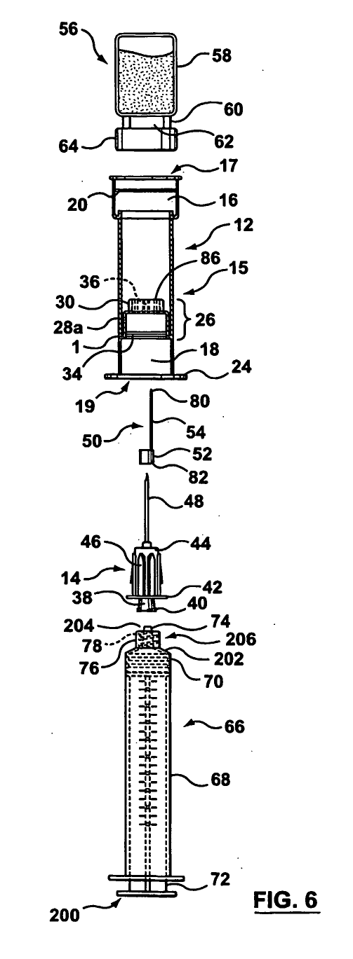

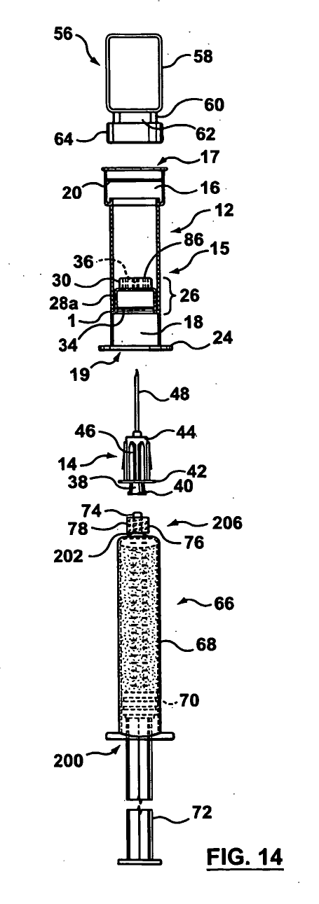

As best seen in FIGS. 6 and 14, a standard pharmaceutical vial 56 generally has a vial body 58, a neck 60 of a reduced diameter compared with the body 58, a penetrable closure 62 typically made from an elastomeric material (e.g. rubber), and a cap 64 to secure the penetrable closure 62 to the pharmaceutical vial 56.

As best seen in FIG. 6, a standard syringe 66 may be a mass-produced moulded plastic syringe having a syringe body 68 being open at one end 200 and having a neck 202 at the opposite end 204. A piston 70 is lodged in the syringe body 68 from the open end 200, the piston 70 being provided with means (not shown) by which a detachable plunger rod 72 may be secured to the piston 70. The neck 202 of the syringe body 68 has a standard needle coupling or “luer lock” 206 comprising a conical spigot 74 with a central passage communicating with the interior of the syringe body 68. The spigot 74 is surrounded by cylindrical sleeve 76 having an internal thread 78 (shown in dotted outline).

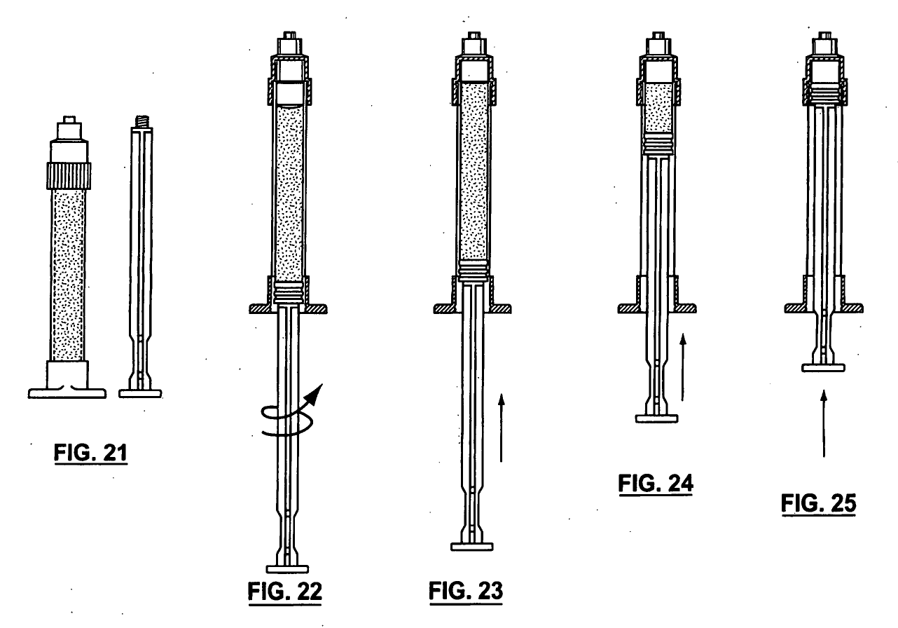

There are other kinds of syringes that are well known in the art, all of which are included with the scope of the present invention. For example, another known syringe is shown in FIGS. 21-25, which has two pistons within the body (one at the neck end and one at the open end) with the pharmaceutical component contained between the two.

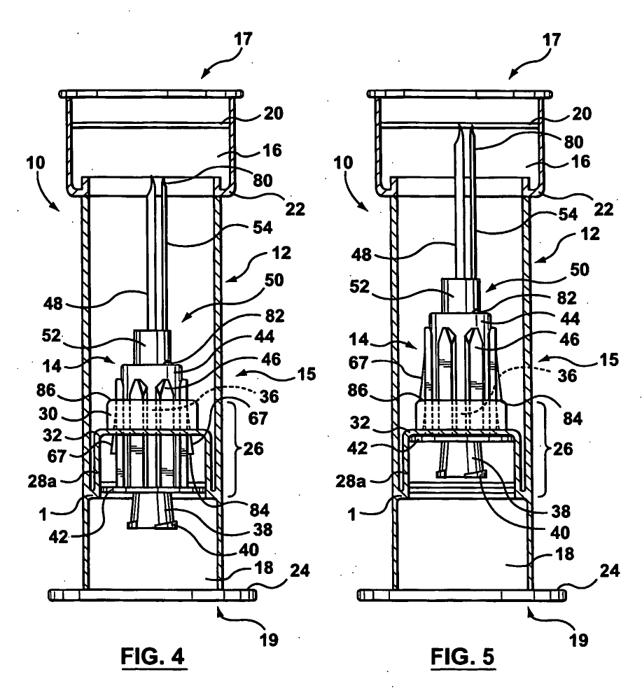

Referring now to FIG. 4, a first embodiment of a transfer assembly made in accordance with the present invention is shown generally at 10. The transfer assembly 10 generally comprises a housing 12 and a protractible luer adapter 14.

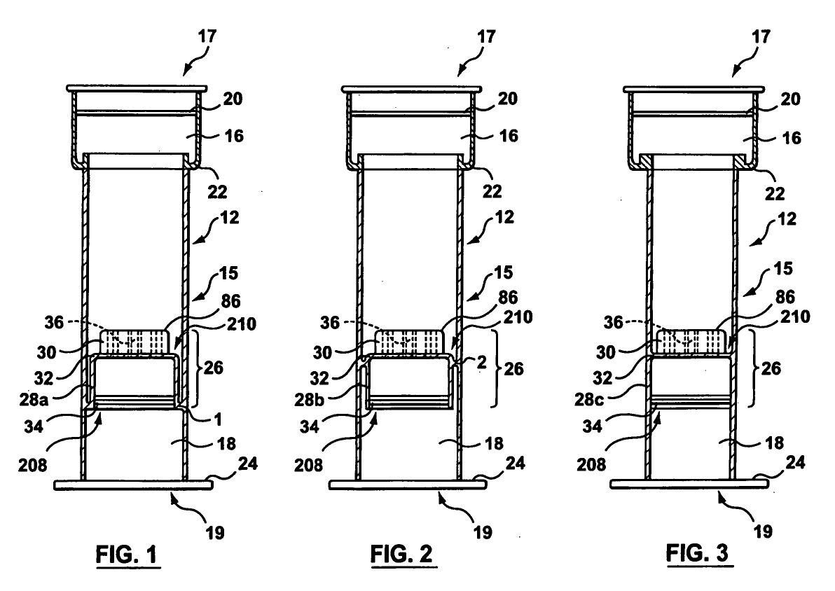

Referring now to FIG. 1, a first embodiment of the housing 12 is shown. The housing 12 may be of any suitable size and shape, and in this embodiment is cylindrical. The housing has central portion 15, a vial socket 16 at one end 17, and a syringe socket 18 at the opposite end 19. The vial socket 16 is appropriately sized and shaped to receive a vial 56 having a penetrable closure 62 and a cap 64 (see FIG. 4), described above. Preferably, the vial socket 16 has an inner annular ridge 20 of slightly smaller dimension than the housing 12 for positively retaining the cap 64 of the vial 56 once it is fully inserted into the vial socket 16 (as shown in FIGS. 8-11 and 16-20). The vial socket 16 is preferably larger in inner diameter than the central portion 15 of the housing 12, thus forming an inner annular shoulder 22 at the juncture of the vial socket 16 and the central portion 15 of the housing 12. In this respect, the vial socket may be sized to accommodate a pharmaceutical vial, for example a vial with a 20 mm finish. The inner annular shoulder 22 serves to limit the degree of insertion of the vial 56 into the vial socket 16. The syringe socket 18 is appropriately sized and shaped to receive a standard syringe 66, described above. The end 19 of the housing 12 preferably has a finger flange 24 to aid in gripping the assembly during operation.

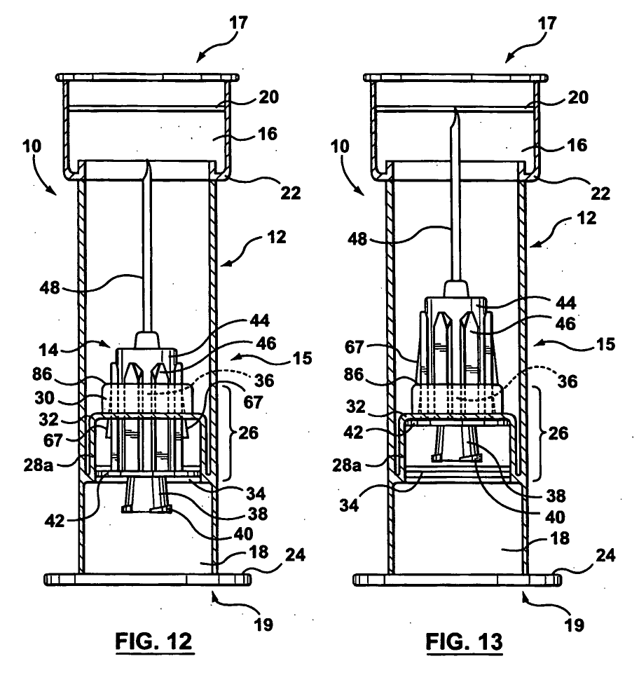

Still referring to FIG. 1, the housing 12 has an inner sleeve 26 that is appropriately sized and shaped to receive the protractible luer adapter 14, which will be described in more detail below. The inner sleeve 26 generally has a first portion 28a and an adjacent second portion 30. In this embodiment, the first portion 28a is connected to the housing 12 by an annular connecting wall 1 that is positioned adjacent a one end 208 of the first portion 28a. The housing 12 has a larger diameter than the first portion 28a, and the first portion 28a has a larger diameter than the second portion 30. An annular shoulder 32 is formed at the juncture between the first portion 28a and the second portion 30. The first portion 28a has an annular detent 34 for positively engaging the protractible luer adapter 14 in a retracted position (as seen in FIGS. 4, 7, 12, 15, and 26) and as will be subsequently described. The inside wall of the second portion 30 has a number of spaced longitudinal ribs 36 (in dotted outline).

Now referring to FIG. 2, a second embodiment of the housing 12 is shown. The second embodiment is the same as the first embodiment, except as described below. Specifically, first portion 28b is connected to the housing 12 by an annular connecting wall 2 that is positioned adjacent top end 210 of the first portion 28b. The first portion 28b may be adapted to flex slightly to facilitate the insertion of the protractible luer adapter 14 into the annular detent 34 for positively engaging the protractible luer adapter 14 in the retracted position.

Now referring to FIG. 3, a third embodiment of the housing 12 is shown. The third embodiment is the same as the first embodiment, except that the first portion 28c is coincident with the wall of the housing 12.

The protractible luer adapter 14 (best seen in FIGS. 6 and 14) has a female luer lock 38 having an external thread 40, a flange 42, a hub 44 having a number of spaced apart longitudinal ribs 46 and at least one protrusion 67 (best seen in FIGS. 4 and 5), and a hollow piercing member 48 coupled to the hub 44. The hollow piercing member 48 may be any suitable device well known in the art, that is capable of penetrating the penetrable closure 62 of the vial 56. In one embodiment the hollow piercing member 48 is a hollow needle such as a standard cannula. In a further embodiment, the hollow piercing member 48 is a plastic needle or spike. In yet a further embodiment, the hollow piercing member 48 is a blunt plastic cannula that cooperates with a pre-slit penetrable closure on the vial (e.g., the INTER-LINK SYSTEM™ which is commercially available from Baxter). The female luer lock 38, hub 44 and hollow piercing member 48 are in fluid communication with each other. The protractible luer adapter 14 may also have a filter media (not shown) disposed between the female luer lock 38 and the hub for filtering fluid as it passes through the protractible luer adapter 14.

The protractible luer adapter 14 is adapted for longitudinal movement within the inner sleeve 26 between a retracted or “unactivated” position (as seen in FIGS. 4, 7, 8, 12, 15, 16 and 18) and an advanced or “activated” position (as seen in FIGS. 5, 9-11, 13, and 17-20). As will be described below in detail, in the retracted position, the hollow piercing member 48 is fully contained within the central portion 15 of the housing 12. In the advanced position, the hollow piercing member 48 protrudes into the vial socket 16 of the housing 12.

Referring to FIG. 4, the flange 42 on the protractible luer adapter 14 is adapted to snap fit into the annular detent 34 on the first portion 28a of the inner sleeve 26 to positively engage the protractible luer adapter 14 and retain the protractible luer adaptor 14 in the retracted or “inactivated” position until activated. Additionally, the flange 42 serves to abut the inner annular shoulder 32 when the protractible luer adaptor 14 is in the advanced or “activated” position, thus limiting the degree of insertion of the syringe into the syringe socket 18 and accordingly the advancement of the hollow piercing member 48 into the vial socket 16. Moreover, while the protractible luer adapter 14 is in the advanced position, the flange 42 serves to substantially contain any fluid which may escape from the vial into the transfer assembly 10. This is particularly important when toxic pharmaceuticals are used. Once assembly 10 has been deployed, the pharmaceutical transferred to the syringe 66, and the syringe 66 has been removed from the assembly 10, the transfer assembly 10 can be safely discarded. In other words, the user will not come into contact with the pharmaceutical component.

The longitudinal ribs 46 located on the hub 44 of the protractible luer adapter 14 are sized and spaced so as to slidingly fit between the longitudinal ribs 36 located on the inner wall of the second portion 30 of the inner sleeve 26. This prevents rotation of the protractible luer adapter 14 with respect to the housing 12 during operation.

The at least one protrusion 67 on the hub 44 is preferably triangular in shape and is appropriately sized to snap fit the protractible luer adapter 14 within the inner sleeve 26 of the transfer assembly 10 when in the advanced position. When the protractible luer adapter 14 is in the advanced position, the bottom portion 84 of the at least one protrusion 67 abuts the top surface 86 of the second portion 30 to prevent the protractible luer adapter 14 from being removed from the inner sleeve 26 of the transfer assembly 10 or being returned to the retracted position (as best seen in FIG. 5). Thus, once the transfer assembly 10 has been deployed into the advanced position, the protractible luer adapter 14 remains fixed in the inner sleeve 26 of the transfer assembly 10. To achieve this preferable configuration where the flange 42 abuts the shoulder 32 of the inner sleeve 26 when the protractible luer adaptor 14 is in the advanced position and the bottom portion 84 of the at least one protrusion 67 engages the top surface 86 of the second portion 30, it will be appreciated that the spacing between the bottom portion 84 of the at least one protrusion 67 and the flange 42 is approximately equal to the spacing between the top surface 86 of the second portion and the shoulder 32. Accordingly, once the syringe 66 is removed from the female luer lock 38 on the protractible luer adapter 14 (by unthreading the two), the rest of the pharmaceutical delivery system, including the empty pharmaceutical vial 56, and the transfer assembly 10 including the protractible luer adapter 14 can be safely discarded. The operation of the transfer assembly will be described in detail below.

Optionally, a venting needle assembly 50 having a base 52 and a venting needle 54 may be used in connection with the transfer assembly 10 as described below. This optional venting needle assembly 50 is shown in FIGS. 4-11. The venting needle assembly 50 provides a vent to prevent any significant pressure increase or decrease in the vial during operation. The venting needle 54 maintains the pressure in the pharmaceutical vial 56 at approximately surrounding atmospheric pressure by permitting air to enter into and escape from the pharmaceutical vial 56 during the transfer of pharmaceutical components from the pharmaceutical vial 56 to the syringe 66 and vice versa. In one embodiment shown in FIGS. 4-11, the tip 80 of the venting needle 54 is in fluid communication with an aperture 82 provided in the base 52. Alternatively, the venting needle 54 may have an opening on its side (not shown). Preferably, the bore of the venting needle 54 is smaller than the bore of the hollow piercing member 48 to prevent leakage through the venting needle 54. This is particularly important if the pharmaceutical is unsafe for the user, for example toxic oncology drugs.

The venting needle assembly 50 is particularly useful when dealing with toxic pharmaceuticals. Specifically, any toxic gases released through the venting needle 54 during operation of the transfer assembly 10 are substantially contained within the transfer assembly 10. The flange 42 of the protractible luer adapter 14 substantially covers the annular shoulder 32 to generally contain any liquid or gases released during operation within the transfer assembly 10.

As stated, the venting needle assembly 50 is preferably optional. Therefore, in a preferred embodiment, the venting needle assembly 50 is removable from the protractible luer adaptor 14. This may be achieved in any known manner. For example, as shown in FIGS. 4-11, the base 52 has a bore (not shown) adapted to slide over the hollow piercing member 48. In another embodiment, the base 52 may be adapted to snap onto the hollow piercing member 48. Other embodiments will be readily recognized by skilled persons in the art.

There are many pharmaceutical delivery systems that can benefit from the incorporation of the venting needle 54. The venting needle 54 is useful for general liquid transfer from the vial into the syringe since the user does not have to force air into the vial prior to aspirating the liquid out of the vial. This helps to prevent accidents that can occur when too much air is forced into the vial (e.g., when the pharmaceutical component sprays through the puncture point made in the penetrable seal and onto the user). The venting needle 54 is particularly preferred for liquid transfer from the vial 56 into the syringe 66 where the liquid contains bubbles that need to be maintained for the end use. For example, some cancer detection imaging systems require the presence of perfluorocarbon bubbles immersed in a liquid. In this case, the venting needle 54 maintains the vial at a substantially constant pressure at all times to prevent the bubbles from bursting under increased or decreased pressure. The venting needle 54 may also be used for reconstitution of a first pharmaceutical component and a second liquid pharmaceutical component in cases where the mixture of the two components results in the production of gaseous by-products. In this case, the venting needle 54 vents the gaseous by-product and prevents the build-up of gases in the vial 56. This helps to prevent accidents that can occur when too much pressure builds up in the vial 56.

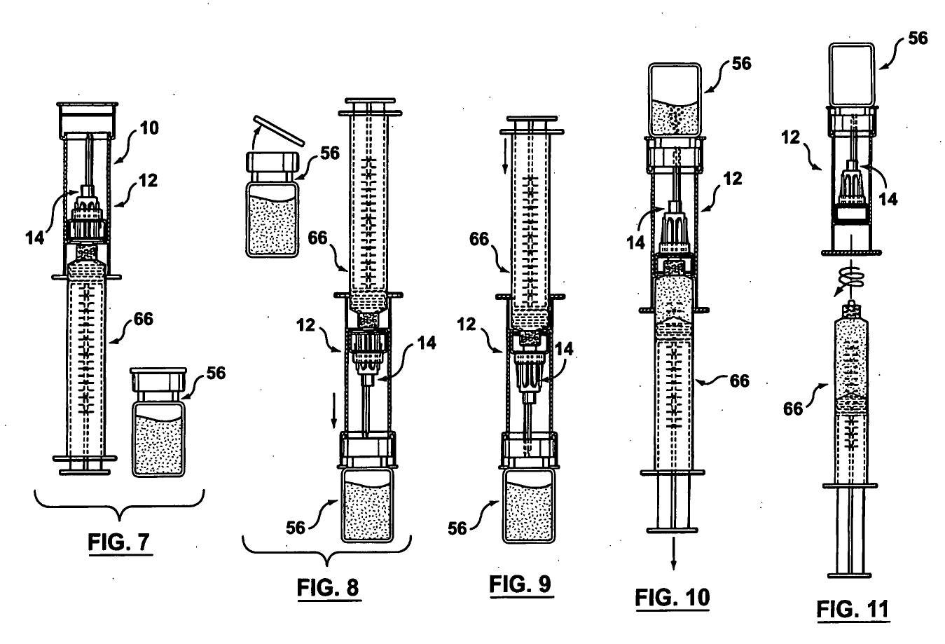

FIGS. 7-11 illustrate the sequential operation of the pharmaceutical delivery system adapted to transfer liquid from the pharmaceutical vial 56 to the syringe 66. In this case, it is preferable to have the venting needle 54 attached to the protractible luer adapter 14 to vent the pharmaceutical vial 56 during operation. An empty mass produced plastic syringe 66 can be pre-attached to the transfer assembly 10 during the manufacturing stage (by threading the conical spigot 74 and cylindrical sleeve 76 having an internal thread 78 onto the external thread 40 of the female luer lock 38 of the protractible luer adapter 14), and the whole device can be sterilized prior to being packaged. Alternatively, the syringe 66 and the transfer assembly 10 may be separately packaged, in which case the user must thread the syringe 66 onto the female luer lock 38 on the protractible luer adaptor 14 prior to use.

Still referring to FIGS. 7-11, the method for deploying the pharmaceutical delivery system generally includes the steps of: (a) removing the cover of the pharmaceutical vial and snap fitting the pharmaceutical vial 56 into the vial socket 16 of the transfer assembly 10 (see FIG. 8); (b) advancing the protractible luer adapter 14 longitudinally within the inner sleeve 26 of the housing 12 from the retracted position wherein there is no fluid communication between the pharmaceutical vial 56 and the syringe 66 to the advanced position wherein the hollow piercing member 48 (and the venting needle 54 if used) pierces the pentrable closure of the vial 56, thus establishing fluid communication between the pharmaceutical vial 56 and the syringe 66 (see FIG. 9) (this may be achieved by similarly advancing the syringe 66, which is coupled to the protractible luer adapter 14, longitudinally within the syringe socket 16); (c) inverting the pharmaceutical delivery system and withdrawing the plunger 72 to aspirate the contents of the pharmaceutical vial 56 into the syringe 66 (see FIG. 10); (e) detaching the syringe 66 from the female luer lock 38 (by unscrewing it) to provide a syringe ready for use (see FIG. 11).

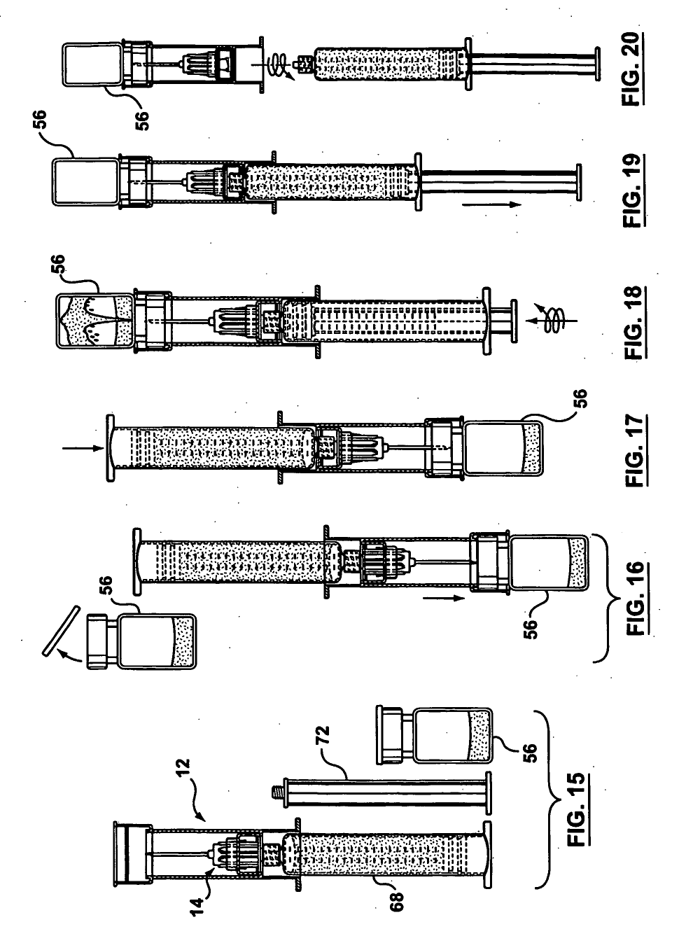

FIGS. 15-20 illustrate the sequential operation of the pharmaceutical delivery system adapted to reconstitute a multi-component pharmaceutical. At least one of the pharmaceutical components is a liquid (e.g., a diluent); usually it will be convenient to locate a liquid component in the syringe but it would be possible to locate a solid component in the syringe. If the liquid pharmaceutical component is prepackaged in the pharmaceutical vial 56 and the solid pharmaceutical component is packaged in the syringe 66, it may be desirable to use the optional venting needle 54. In this case, the venting needle 54 obviates the need to provide an air volume in the syringe body 68 that is sufficient to force a given volume of air into vial prior to aspirating the contents. Additionally, it might be desirable to use the optional venting needle 54 if the solid contained in the pharmaceutical vial 56 is under a high vacuum. Alternatively, the venting needle can be removed if it is not required.

Still referring to FIGS. 15-20, the method for deploying the pharmaceutical delivery system using a syringe prefilled with a liquid (e.g., a diluent) typically comprises the steps of: (a) removing a protective cap (not shown) from the neck of the syringe 66 and threading the syringe 66 onto the female luer lock 38 (as shown in FIG. 15); (b) removing the cover of the pharmaceutical vial 56 containing a second pharmaceutical component and snap fitting the pharmaceutical vial 56 into the vial socket 16 of the transfer assembly 10 (see FIG. 16); (c) advancing the syringe 66 and thus the protractible luer adapter 14 longitudinally within the inner sleeve 26 of the housing 12 from the retracted position to the advanced position wherein the tip of the hollow piercing member 48 penetrates the penetrable closure 62 on the vial 56 to create fluid communication between the pharmaceutical vial 56 and the syringe 66 (see FIG. 17); (d) inverting the pharmaceutical delivery system, attaching the plunger rod 72 to the piston 70, and injecting the liquid (e.g., a diluent) from the syringe into the pharmaceutical vial (see FIG. 18);

(e) swirling the pharmaceutical delivery system to dissolve, dilute or suspend the liquid component into the second pharmaceutical component; (f) withdrawing the plunger 72 to aspirate the contents of the pharmaceutical vial 56 into the syringe 66 (see FIG. 19); and (g) detaching the syringe 66 from the female luer lock 38 (by unthreading the two) to provide a syringe ready for use (see FIG. 20).

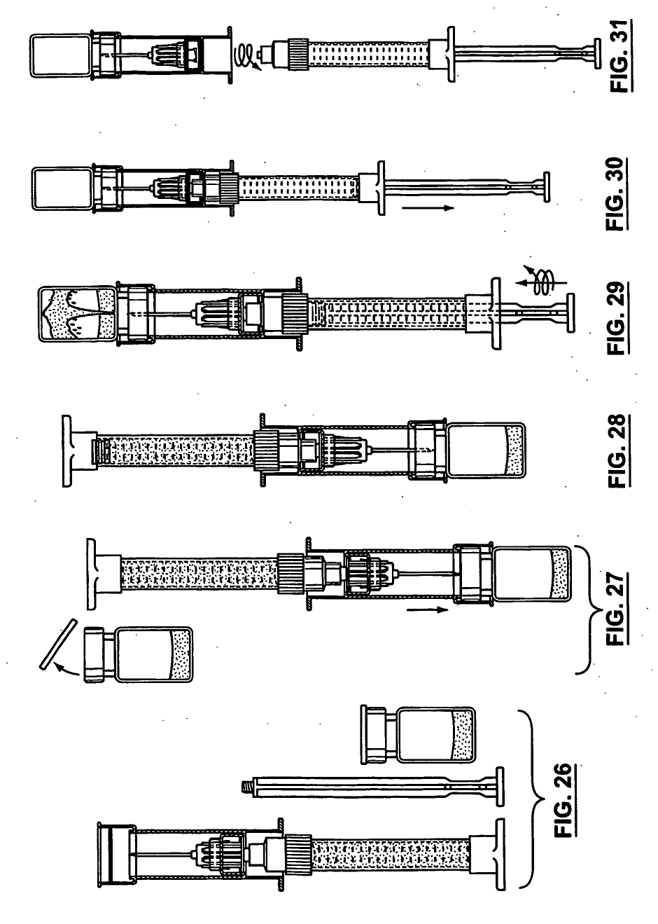

FIGS. 26-31 illustrate the sequential operation of a pharmaceutical delivery system according to another aspect of the invention using the type of syringe shown in FIGS. 21-25 (where the syringe has two pistons and contains a pre-packaged pharmaceutical component). The method of operation is substantially the same as described with respect to FIGS. 15-20, except as described below. This pre-filled syringe can be pre-attached to the transfer assembly 10 during the manufacturing stage (by threading the conical spigot 74 and cylindrical sleeve 76 having an internal thread 78 onto the external thread 38 of the female luer lock 38 of the protractible luer adapter 14), and the whole device can be sterilized prior to being packaged. Accordingly, the user does not have to attach the syringe 66 onto the transfer assembly 10.

Thus any pre-filled syringe can be pre-attached in the manner described above, provided the primary closures are not opened or breached before attachment to the protractible luer adapter 14. An example of such a syringe is described in U.S. Pat. No. 3,967,759 by Baldwin which is incorporated by reference. Other piston by-pass syringes that are well known in the syringe art can also be used.

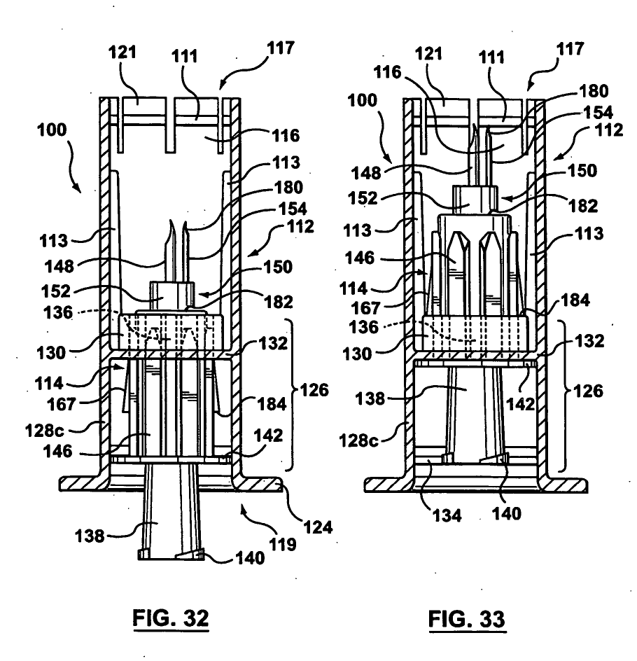

Referring now to FIGS. 32 and 33, a second embodiment of a transfer assembly made in accordance with the present invention is shown generally at 110. This embodiment has many similarities with the embodiments previously described and which will not be repeated in detail. The transfer assembly 110 generally comprises a housing 112 and a protractible luer adapter 114.

The housing 112 may be of any suitable size and shape, and in this embodiment is cylindrical. The housing has central portion 115, a vial socket 116 at one end 117, and an opposite open axial end 119. The vial socket 116 is appropriately sized and shaped to receive a standard pharmaceutical vial 56 having a penetrable closure 62 and a cap 64, described above. Preferably, the vial socket 116 has a plurality of latches 111 (in the form of an annular ridge around the inner circumference of the vial socket 116, which is divided by a plurality of longitudinal slots 121). The slots 121 permit the vial socket 116 some flexibility to facilitate insertion of the pharmaceutical vial 56. The latches 11 positively retain the cap 64 of the vial 56 once it is fully inserted into the vial socket 116. The vial socket 116 is preferably equal in inner diameter to the central portion 115 of the housing 112. By this respect, the vial socket 116 may be sized to accommodate a pharmaceutical vial, for example a vial with a 13 mm finish. The housing 112 is provided with at least one longitudinal rib 113 that serves to limit the degree of insertion of the vial 56 into the vial socket 116.

In this embodiment the housing 112 does not include a syringe socket. Instead, the protractible luer adapter 114 extends past the end 119 of the housing. This allows the transfer assembly 110 to be coupled with any type of syringe known in the art that is provided with a standard luer lock, irrespective of the diameter of the syringe barrel. For example, the transfer assembly 110 can be coupled with a BD READYFILL™ glass syringe, a BD HYPAK™ glass syringe, a BUNDER GLAS RTF™ syringe, a BD STERIFILL™ plastic syringe, a SCHOTT TOPAC™ plastic syringe, Abbott ANSWER™ plastic syringe, or the like. The end 119 of the housing 112 preferably has a finger flange 124 to aid in gripping the assembly during operation.

The housing 112 has an inner sleeve 126 that is appropriately sized and shaped to receive the protractible luer adapter 114, which will be described in more detail below. The inner sleeve 126 generally has a first portion 128c and an adjacent second portion 130. In this embodiment, the first portion 128c is coincident with the housing 12. The first portion 128c has a larger diameter than the second portion 130. An annular shoulder 132 is formed at the juncture between the first portion 128c and the second portion 130. The first portion 128c has an annular detent 134 for positively engaging the protractible luer adapter 114 in a retracted position (as seen in FIG. 32). The inside wall of the second portion 130 has a number of spaced longitudinal ribs 136 as best seen in FIG. 32 (in dotted outline.)

The protractible luer adapter 114 has a female luer lock 138 having an external thread 140, a flange 142, a hub 144 having a number of spaced apart longitudinal ribs 146 and at least one protrusion 167 and a hollow piercing member 148 coupled to the hub 144. The female luer lock 138, hub 144 and hollow piercing member 148 are in fluid communication with each other.

The protractible luer adapter 114 is adapted for longitudinal movement within the inner sleeve 126 between a retracted or “unactivated” position (as seen in FIG. 32) and an advanced or “activated” position (as seen in FIG. 33). In the retracted position, the hollow piercing member 148 is fully contained within the central portion 115 of the housing 112. In the advanced position, the hollow piercing member 148 protrudes into the vial socket 116 of the housing 112.

Optionally, a venting needle assembly 150 having a base 152 and a venting needle 154 may be used in connection with the transfer assembly 110 as described above.

The operation of this embodiment is substantially the same as for the previously described embodiments.

While the above description constitutes the preferred embodiments, it will be appreciated that the present invention is susceptible to modification and change without departing from the fair meaning of the proper scope of the accompanying claims.

1. An assembly for transferring a liquid between a vial and a syringe, comprising:
a) a housing having a central portion, the housing being open at one end and having a vial socket at the other opposite end adapted to receive and retain a vial having a penetrable closure;
b) a sleeve located within the central portion of the housing, the sleeve having a first portion, a second portion adjacent the first portion, and a shoulder between the first portion and the second portion;
c) a protractible luer adaptor with a central hub having a first axial end and a second axial end, the first axial end having mounted thereon a piercing member having a bore and a tip having an opening and the second axial end having an engaging member for releasably engaging a syringe, the piercing member, the central hub, and the engaging member being in fluid communication with one another;
d) the protractible luer adaptor being longitudinally slidable within the sleeve between a retracted position where the tip of the piercing member is substantially contained within the central portion of the housing and an advanced position where the tip of the hollow piercing member extends into the vial socket.

2. An assembly according to claim 1 further comprising a venting needle assembly releasably mounted on the first axial end of the protractible luer adaptor to provide a passageway for gas to flow between a vial retained in the vial socket and the assembly when the protractible luer is in the advanced position.

3. An assembly according to claim 2 wherein the venting needle assembly comprises a needle having a bore and a tip with a first opening, and a base having a second opening, the first and second openings being in fluid communication with one another.

4. An assembly according to claim 3 wherein the diameter of the bore of the venting needle is smaller than the diameter of the bore of the piercing member.

5. An assembly according to claim 1 further comprising a syringe socket at the open end of the housing for receiving a syringe.

6. An assembly according to claim 1 further comprising a retaining member in the vial socket for retaining a vial within the vial socket.

7. An assembly according to claim 6 wherein the retaining member comprises an annular ridge on the interior surface of the vial socket, the annular ridge having a smaller diameter than the diameter of the vial socket.

8. An assembly according to claim 6 wherein the retaining member comprises a plurality of latches provided in the vial socket.

9. An assembly according to claim 1 further comprising a shoulder between the vial socket and the central portion of the housing to limit the degree of insertion of a vial in the housing.

10. An assembly according to claim 1 wherein the housing includes at least one rib on an interior surface of the housing to limit the degree of insertion of a vial in the housing.

11. An assembly according to claim 1 wherein the interior surface of the first portion includes a detent engaging and retaining the protractible luer adaptor in the retracted position.

12. An assembly according to claim 11 wherein the protractible luer adaptor includes a flange adjacent the second end and the detent is configured to receive the flange therein.

13. An assembly according to claim 1 further comprising a plurality of longitudinal ribs on an interior surface of the second portion of the sleeve that matingly engage a plurality of longitudinal ribs on the central hub of the protractible luer adaptor to prevent rotation of the protractible luer adaptor with respect to the housing during operation.

14. An assembly according to claim 1 wherein the protractible luer adaptor includes a flange adjacent the second end, the flange abutting the shoulder between the first portion and the second portion while in the advanced position to limit the advancement of the tip of the piercing member into the vial socket.

15. An assembly according to claim 14 wherein the diameter of the flange is substantially equal to the inner diameter of first portion of the sleeve to provide a fluid seal therewith when the protractible luer adaptor is in the advanced position.

16. An assembly according to claim 1 further comprising at least one protrusion an exterior surface of the protractible luer adaptor, the at least one protrusion having a bottom edge and a side edge, the bottom edge abutting the top surface of the second portion of the sleeve while in the advanced position to prevent the protractible luer adaptor from being removed from the sleeve.

17. An assembly for use with a syringe having a body, a neck end, and a hollow cannula, the assembly comprising a needle having a tip with a first opening, a base with a second opening, and a central bore extending between the first and second openings, the base adapted to be releasably mounted the neck end of the syringe.
